(19) United States
(12) Patent Application Publication
(10) Pub. No.: US 2015/0079202 A1
(43) Pub. Date: Mar. 19, 2015

(54) USE OF PATCHOULI EXTRACT IN THE PREPARATION OF COMPOSITIONS WITH AN ANTI-MICROORGANISM EFFECT

(71) Applicant: ONENESS BIOTECH CO., Ltd, Taipei City (TW)

(72) Inventors: HUI-CHUN CHEN, Taipei City (TW); JEN-WEI CHEN, Taipei City (TW); HSIAO-CHU HUANG, Taipei City (TW); WEI-LUEN CHANG, Taipei City (TW)

(21) Appl. No.: 14/475,888

(22) Filed: Sep. 3, 2014

(30) Foreign Application Priority Data
Sep. 3, 2013 (TW) 102131723

(51) Int. Cl. A61K 36/53; A61Q 19/00; A61K 31/60; A61K 45/06; A61K 36/23; A61K 8/97; A61Q 19/10

(52) U.S. Cl. CPC A61K 36/53; A61K 8/97; A61Q 19/00; A61Q 19/10; A61K 45/06; A61K 36/23; A61K 31/60
USPC 424/745

(57) ABSTRACT

The present invention provides a method or composition for inhibiting the infection of a microorganism in a subject, using a Plectranthus amboinicus (Lour.) Spreng extract. Also provided is a method or composition for treating or preventing acne vulgaris caused by microorganisms, particularly Propionibacterium acnes, Staphylococcus aureus, and Pseudomonas aeruginosa.

### CROSS REFERENCE TO RELATED APPLICATIONS

This application claims benefit under 35 U.S.C 119 (a) of Taiwan Application No. 102131723, filed Sep. 3, 2013, the entire content of which are incorporated by reference herein.

### FIELD OF THE INVENTION

The present invention provides a method for inhibiting the infection of a microorganism, comprising administering the subject with a composition comprising an effective amount of a Plectranthus amboinicus (Lour.) Spreng extract.

### BACKGROUND OF THE INVENTION

Acne vulgaris, commonly known as acne, is a disease with inflamed hair follicles and sebaceous glands. More than 85% adults have experienced acne when they were growing up. Based on different symptoms, acne can be divided into non-inflammatory acne such as comedones, and inflammatory acne such as cysts or inflamed hair follicles. Among them, it is the most difficult to cure inflammatory acne induced by the overgrowth of bacteria caused by seborrhoea.

It is commonly known that inflammatory acne induced by bacteria can be treated through external medications and oral medications. For external medications, antibiotic creams or liquids (such as Cleocin or Fucidin) are mainly used to inhibit the growth of Propionibacterium acnes to alleviate inflammation. For oral medications, oral antibiotics are used as the first-line medications for acne treatment. It is known that the antibiotics for oral medications include tetracycline, erythromycin, and sulfonamides, which are effective in reducing the generation of non-esterified fatty acids and thus providing an anti-inflammatory effect, but not in killing bacteria.

It is desirable to utilize natural ingredients to inhibit those bacteria causing inflammatory acne and thus reduce the use or intake of antibiotics.

### BRIEF SUMMARY OF THE INVENTION

Accordingly, the present invention provides a method for inhibiting the infection of a microorganism in a subject, comprising administering the subject with a composition comprising an effective amount of a Plectranthus amboinicus (Lour.) Spreng extract.

According to the invention, the microorganism comprise bacterium. In one embodiment of the invention, said bacterium is Propionibacterium sp., Staphylococcus sp., or Pseudomonas sp. In one specific example, said bacterium is Propionibacterium acnes, Staphylococcus aureus, or Pseudomonas aeruginosa.

In one example of the invention, the Plectranthus amboinicus (Lour.) Spreng extract is obtained by extracting the plant or dried powder of Plectranthus amboinicus (Lour.) Spreng in a solvent to obtain a crude extract, and eluting the resulted crude extract by an alcohol solution at a concentration of at least 50%, preferably 50%˜95% and most preferably 70%˜95%. In one specific example, the alcohol solution is a methanol or ethanol solution.

According to the invention, the Plectranthus amboinicus (Lour.) Spreng extract may be a combination of a first extract obtained by extracting the plant or dried powder of Plectranthus amboinicus (Lour.) Spreng in a solvent to obtain a crude extract, and eluting the resulted crude extract by 50%˜95% alcohol solution, and a second extract obtained by extracting the plant or dried powder of Plectranthus amboinicus (Lour.) Spreng in a solvent to obtain a crude extract, and eluting the resulted crude extract by 70%˜95% alcohol solution. Also, said 50%˜95% alcohol solution may be a methanol or ethanol solution, and said 70%˜95% alcohol solution may be a methanol or ethanol solution.

In one particular example of the invention, the Plectranthus amboinicus (Lour.) Spreng extract is obtained by extracting the plant or dried powder of Plectranthus amboinicus (Lour.) Spreng in a solvent to obtain a crude extract, and eluting the resulted crude extract by an alcohol solution, followed by elution with ethyl acetate. Said alcohol solution may be a methanol or ethanol solution at a concentration of at least 50%, preferably 50%˜95% and most preferably 70%˜95%.

In another aspect, the present invention provides the use of the Plectranthus amboinicus (Lour.) Spreng extract for manufacturing a medicament for inhibiting the infection of a microorganism in a subject.

In one further aspect, the present invention provides a composition or pharmaceutical composition for inhibiting the infection of a microorganism comprising an effective amount of the Plectranthus amboinicus (Lour.) Spreng extract.

In one yet aspect, the present invention provides a method for inhibiting the infection of a microorganism in a subject, comprising tadministering the subject with a composition in an amount effective in inhibiting the infection of a microorganism, wherein the composition comprises a Plectranthus amboinicus (Lour.) Spreng extract, wherein said composition also comprises an appropriate excipient in order to be prepared in the form of an external medicine, a cosmetic product or a cleanser.

In one further yet aspect, the present invention provides a method for treating or preventing acne vulgaris in a subject, comprising administering the subject with a composition comprising an amount effective of a Plectranthus amboinicus (Lour.) Spreng extract.

In one example of the invention, the composition may be Plectranthus amboinicus (Lour.) Spreng extract in combination of another antimicrobial agent, an anti-inflammatory drug, a cleaning or skin care component, or combinations thereof. the composition may be prepared in the form of an external medicine, a cosmetic product or a cleanser.

In one particular example of the present invention, the composition comprises a Plectranthus amboinicus (Lour.) Spreng extract in combination of a Centella Asiatica extract and/or salicylic acid. The composition may be prepared in the form of an external medicine, a cosmetic product or a cleanser.

Those and other aspects of the present invention may be further clarified by the following descriptions and drawings of preferred embodiments. Although there may be changes or modifications therein, they would not betray the spirit and scope of the novel ideas disclosed in the present invention.

### DETAILED DESCRIPTION OF THE INVENTION

Unless defined otherwise, all technical and scientific terms used herein have the same meanings as commonly understood by one of skill in the art to which this invention belongs.

Unless clearly specified herein, meanings of the articles “a,” “an,” and “said” all include the plural form of “more than one.” Therefore, for example, when the term “a component” is used, it includes multiple said components and equivalents known to those of common knowledge in said field.

The term “inhibit” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “infection” as used herein refers to presence of a microorganism, in or on a subject, which, if its growth were inhibited, would result in a benefit to the subject.

The term “microorganism” includes prokaryotic and eukaryotic microbial species from archaea, bacteria or eucarya. The eukaryotic microbial species include yeast and filamentous fungi, protozoa, algae, or higher protista.

As used herein, the term “subject” refer to a human or a non-human mammal, such as a patient, a companion animal (e.g., dog, cat, and the like), a farm animal (e.g., cow, sheep, pig, horse, and the like) or a laboratory animal (e.g., rat, mouse, rabbit, and the like).

As used herein, the term “treating” may include prophylaxis of the specific infection, injury, disease, disorder, or condition, or alleviation of the symptoms associated with a specific infection, injury, disease, disorder, or condition and/or preventing or eliminating said symptoms if specifically stated as being a prophylactic treatment. “Treating” is used interchangeably with “treatment” herein.

The term “effective amount” used herein refers to a concentration of the composition that can effectively treat or prevent acne vulgaris; it can be adjusted according to the means of administration and therapeutic conditions, including age, body weight, symptoms, therapeutic effects, means of administration, and therapeutic time.

The term “vehicle or carrier” or “pharmaceutically acceptable vehicle or carrier” used herein refers to diluents, excipients or the like used in pharmaceutics, including those well known to one of ordinary skills in the pharmaceutical industry.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The present invention provides a new use of Plectranthus amboinicus extract for inhibiting the infection of a microorganism. According to the present invention, the Plectranthus amboinicus in any forms may be used, including but not limited to, a fresh or dried plant, preferably in a form of powder.

In the invention, the Plectranthus amboinicus extract is obtained by extracting the plant or dried powder of Plectranthus amboinicus (Lour.) Spreng in a solvent to obtain a crude extract, and eluting the resulted crude extract by alcoholic solution. The crude extract of the plant can be obtained by any of the known processes, for example, extracting in a solvent, including but not limited to, alcoholic solution, such as the 95% ethanol solution. According to this invention, the alcoholic solution for elution may be methanol or preferably ethanol. In another embodiment, the process comprises further a step of eluting the resulted crude extract by an alcohol solution at a concentration of at least 50%, preferably 50%˜95% and most preferably 70%˜95%. In one specific example, the alcohol solution is a methanol or ethanol solution. In particular, the Plectranthus amboinicus extract can be prepared as mentioned in U.S. patent application Ser. No. 13/680,689, the entire content of which are incorporated by reference herein.

According to the present invention, the composition of the present invention may comprise further another antimicrobial agent, an anti-inflammatory drug, a cleaning or skin care component, or combinations thereof. The composition may be prepared in the form of an external medicine or a cosmetic product or a cleanser. In one embodiment, the composition may comprise further a Centella Asiatica extract, or/and salicylic acid. Commonly used vehicle or carrier may be added with any known techniques. Also, an effective amount of the composition of the present invention may be supplemented with a pharmaceutically acceptable vehicle or carrier to be formulated into a medicine based on techniques or methods commonly used in the pharmaceutical field.

According to the invention, it is unexpectedly found that the Plectranthus amboinicus (Lour.) Spreng extract is effective in inhibition of the infection of a microorganism, particularly a bacterium, such as Propionibacterium sp., Staphylococcus sp., or Pseudomonas sp., including a bacterium selected from the group consisting of Propionibacterium acnes, Staphylococcus aureus, and Pseudomonas aeruginosa.

Accordingly, the present invention provides a composition or method for treating or preventing acne vulgaris, in which the composition is in the form of an external formula, and comprises the Plectranthus amboinicus (Lour.) Spreng extract, and optionally a Centella Asiatica extract.

The present invention is explained in the above description of the invention and the following examples, which are not intended for limiting the scope of the present invention.

### EXAMPLE 1

### Preparation of Plectranthus amboinicus (Lour.) Spreng Extract with a 50%˜95% Ethanol Solution

A required amount of dry Plectranthus amboinicus (Lour.) Spreng medical powder was added into a 95% ethanol solution for extraction. The dregs were obtained by filtration, and extracted for a second time with another 95% ethanol solution and filtered. Filtrates from the two extractions were combined and put through reduced-pressure concentration until about 1/20 of the original filtrate volume was left. The thus obtained concentrated extract was ready for column chromatography.

The concentrated extract obtained above was put through column chromatography with the chromatography resin HP-20. Specifically, the obtained concentrated extract was added into RO water, mixed thoroughly, and introduced into the column for chromatography. Then, RO water was introduced into the column for elution and the eluate was collected, followed by elution by introducing 4-times the column volume of a solution of RO water and 95% ethanol (volume ratio 1:1) into the column and collecting the eluate. The eluate was put through reduced-pressure concentration until a small amount was left, which was freeze-dried to obtain a Plectranthus amboinicus (Lour.) Spreng extract to be used as the control group of elution with a 0%-50% ethanol solution.

Then, 4-times the column volume of a 95% ethanol solution was introduced into the column for elution, and the eluate was collected, put through reduced-pressure concentration, and dried to further obtain another Plectranthus amboinicus (Lour.) Spreng extract which was numbered F3. This was the extract eluted with a 50%˜95% ethanol solution. Further, ethyl acetate was introduced into the column for elution, and the eluate was collected, put through reduced-pressure concentration, and dried to obtain a Plectranthus amboinicus (Lour.) Spreng extract eluted with ethyl acetate.

### EXAMPLE 2

### Preparation of Plectranthus amboinicus (Lour.) Spreng Extract Eluted with a 70%˜95% Ethanol Solution

Extraction was performed according to the steps in Example 1 to obtain a concentrated extract which was ready for column chromatography. The obtained concentrated extract was put through column chromatography with the chromatography resin HP-20. The obtained concentrated extract was added into RO water, mixed thoroughly, and introduced into the column for chromatography, followed by introducing RO water into the column for elution and collecting the eluate. Then, a solution of RO water and 95% ethanol (volume ratio 3:7) was introduced into the column for elution. The eluate was collected, reduced to a small amount by reduced-pressure concentration, and freeze-dried to obtain a Plectranthus amboinicus (Lour.) Spreng extract. Then, a 95% ethanol solution was introduced into the column for elution, and the eluate was collected, put through reduced-pressure concentration, and dried to further obtain another Plectranthus amboinicus (Lour.) Spreng extract which was numbered F4. This was the extract eluted with a 70%˜95% ethanol solution. Further, ethyl acetate was introduced into the column for elution, and the eluate was collected, put through reduced-pressure concentration, and dried to obtain a Plectranthus amboinicus (Lour.) Spreng extract eluted with ethyl acetate.

### EXAMPLE 3

### In vitro Antibiotic Experiments

Microorganism infection plays an important role in the course of acne vulgaris, wherein Propionibacterium acnes and Staphylococcus aureus have a more severe and most common influence. This experiment utilized Propionibacterium acnes and Staphylococcus aureus to evaluate the bacteria-inhibiting abilities of the Plectranthus amboinicus (Lour.) Spreng extract of the present invention and compositions prepared using the same.

The composition of the present invention as used for experiment is the Plectranthus amboinicus (Lour.) Spreng extract as prepared above, consisting of F3 (the extract eluted with a 50%˜95% ethanol solution) and F4 (the extract eluted with a 70%˜95% ethanol solution), and a formulation comprising the the Plectranthus amboinicus (Lour.) Spreng extract (the “Dou-con” formulation), comprising F3 and F4, a Centella Asiatica extract, salicylic acid, and a pharmaceutically acceptable vehicle or carrier. The amount of a test sample as used is 1% in a test well containing a tested stain of a microorganism (2-8×10⁵ CFU/ml), Propionibacterium acnes or Staphylococcus aureus. After culturing for 1 to 2 days under a controlled temperature and medium conditions, the opaqueness was determined with a spectrophotometer (wavelength=600 nm) to examine the growth of the microorganisms. A “+” sign means the growth was inhibited, while a “−” sign means opaque/microorganism growth not affected. The results obtained are shown in Table 1 below.

TABLE 1
Bacteria-inhibiting abilities of the test samples

| Test Samples | Propionibacterium acnes | Staphylococcus aureus |
|---|---|---|
| Patchouli Extract 1% | + | + |
| Dou-con Formulation | + | + |
| 1% | | |

Based on the results of the experiment, it is concluded that either of the Plectranthus amboinicus (Lour.) Spreng extract and Dou-con Formulation according to the invention has an effect of inhibition of the total growth of the acne-causing bacteria stains, including Propionibacterium acnes and Staphylococcus aureus.

I/We claim:

1. A method for inhibiting the infection of a microorganism in a subject, comprising administering the subject with a composition comprising an effective amount of a Plectranthus amboinicus (Lour.) Spreng extract, which is obtained by extracting the plant or dried powder of Plectranthus amboinicus (Lour.) Spreng in a solvent to obtain a crude extract, and eluting the resulted crude extract by an alcohol solution at concentration of at least 50%.

2. The method of claim 1, wherein the microorganism is a bacterium.

3. The method of claim 2, wherein the bacterium is Propionibacterium sp., Staphylococcus sp., or Pseudomonas sp.

4. The method of claim 2, wherein the bacterium is selected from the group consisting of Propionibacterium acnes, Staphylococcus aureus, and Pseudomonas aeruginosa.

5. The method of claim 1, wherein the alcohol solution is a 50%˜95% alcohol solution.

6. The method of claim 5, wherein the alcohol solution is a methanol or ethanol solution.

7. The method of claim 1, wherein the alcohol solution is a 70%˜95% alcohol solution.

8. The method according to claim 7, wherein the alcohol solution is a methanol or ethanol solution.

9. The method according to claim 1, wherein the solvent is a 95% ethanol solution.

10. The method of claim 1, wherein the Plectranthus amboinicus (Lour.) Spreng extract is a combination of a first extract obtained by extracting the plant or dried powder of Plectranthus amboinicus (Lour.) Spreng in a solvent to obtain a crude extract, and eluting the resulted crude extract by 50%˜95% alcohol solution, and a second extract obtained by extracting the plant or dried powder of Plectranthus amboinicus (Lour.) Spreng in a solvent to obtain a crude extract, and eluting the resulted crude extract by 70%˜95% alcohol solution.

11. The method according to claim 10, wherein the solvent is a 95% ethanol solution.

12. The method of claim 10, wherein the alcohol solution is a methanol or ethanol solution.

13. The method of claim 1, wherein the Plectranthus amboinicus (Lour.) Spreng extract is obtained by a process further comprising a step of eluting with ethyl acetate.

14. The method of claim 5, wherein the Plectranthus amboinicus (Lour.) Spreng extract is obtained by extracting the plant or dried powder of Plectranthus amboinicus (Lour.) Spreng in a solvent to obtain a crude extract, and eluting the resulted crude extract by 50%˜95% methanol or ethanol solution, followed by eluting with ethyl acetate.

15. The method of claim 7, wherein the Plectranthus amboinicus (Lour.) Spreng extract is obtained by extracting the plant or dried powder of Plectranthus amboinicus (Lour.) Spreng in a solvent to obtain a crude extract, and eluting the resulted crude extract by 70%˜95% methanol or ethanol solution, followed by eluting with ethyl acetate.

16. The method of claim 1, wherein the composition is prepared in the form of an external medicine, a cosmetic product or a cleanser.

17. The method according to claim 1, wherein the composition is effective in treating or preventing acne vulgaris.

18. The method according to claim 17, wherein the composition is in combination of an antimicrobial agent, an anti-inflammatory drug, a cleaning or skin care component, or combinations thereof.

19. The method according to claim 17, wherein the composition further comprises a Centella Asiatica extract.

20. The method according to claim 17, wherein the composition further comprises salicylic acid.
